(19) United States
(12) Patent Application Publication
(10) Pub. No.: US 2004/0152897 A1
(43) Pub. Date: Aug. 5, 2004

(54) Synthesis of indolizines

(75) Inventors: Lijun Sun, Harvard, MA (US); Keizo Koya, Chestnut Hill, MA (US); Zhi-Qiang Xia, Dedham, MA (US); Teresa Przewloka, Tewksburu, MA (US); Shijie Zhang, Nashua, NH (US); Mitsunori Ono, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Synta Pharmaceuticals Corp., Lexington, MA (US)

(21) Appl. No.: 10/660,358

(22) Filed: Sep. 11, 2003

Related U.S. Application Data
(60) Provisional application No. 60/410,679, filed on Sep. 13, 2002.

(51) Int. Cl. C07D 487/02

(52) U.S. Cl. 546/138; 548/453

(57) ABSTRACT

Disclosed are methods of preparing substituted indolizines represented by the following formula:

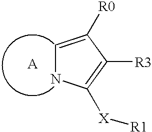

comprising reacting a substrate represented by the following formula:

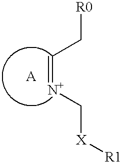

with either the cyclization reagent of the following formula:

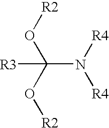

or, a reagent prepared by reacting the compound represented the formula below with an alkylating agent:

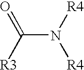

The variables in the above formulas are defined herein.

### RELATED APPLICATIONS

This application claims the benefit of U.S. Provisional Application Serial No. 60/410,679, filed Sep. 13, 2002, the entire teachings of which are incorporated herein by reference.

### BACKGROUND OF THE INVENTION

It has recently been disclosed in U.S. Published Application No. 20030153759 filed Sep. 13, 2002, the entire teachings of which are incorporated herein by reference, that 1-glyoxylamide indolizines, represented by structural formula I, possess anticancer activity, even when administered individually against multi-drug resistant tumors:

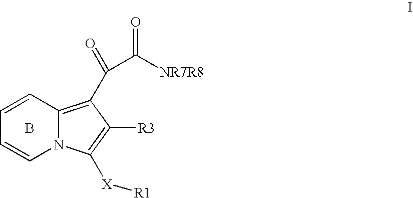

The variables in Formula I are defined below.

Furthermore, other substituted indolizine compounds with a range of pharmacological activity have been reported, for example, for septic shock (WO 96/03383, WO 99/51605), stroke (WO 98/47507), disorders associated with apoptosis (WO99/24033), and isechemic reflow failure (WO 00/021563). There is therefore a need for new synthetic methods that efficiently produce pharmacologically active indolizines, and minimize or eliminate unwanted isomers and waste products.

3-Acyl indolizines, represented in structure II, are key intermediates in the preparation of many pharmacologically active indolizines, including 1-glyoxylamide indolizines:

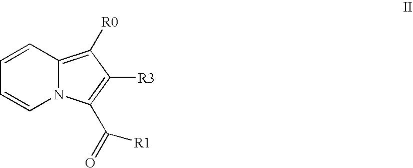

Unfortunately, synthetic routes towards substituted indolizine intermediates in the prior art result in low overall yields of the 3-acyl isomer.

For example, Copar, A.; Stanovnik, B.; Tisler, M. J. Heterocyclic Chem. 30, 1993, 1577-1579 disclose the preparation of acyl indolizines by reacting a substrate, shown below as 1-acetonyl-2-methylpyridinium chloride (1), with a cyclization reagent, specifically dimethyl formamide dimethyl acetal (2):

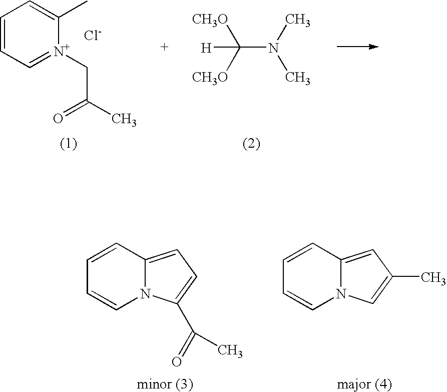

Unfortunately, in such reactions, 3-acyl indolizines are formed as minor by-products in yields ranging from 0 to 20%.

The ability to synthesize 3-acyl indolizines economically and in high yield is a prerequisite to making pharmacologically active indolizines viable as drug candidates. This is essential to bringing new medicines to the public, including anticancer compounds such as I. Herein is disclosed significantly improved synthesis of substituted indolizine compounds.

### SUMMARY OF THE INVENTION

It has now been found that 3-acyl indolizines such as structure II can be prepared in high yield by the use of new, sterically hindered cyclization reagents. The surprising and significant effect of using these new cyclization reagents is that the prior art product distribution is reversed—the 3-acyl indolizine is the major cyclization product and the 2-acyl indolizine is the minor product or is not observed at all. Typically, yields of the 3-acyl indolizine are 70% or greater (see Examples 1 and 2). For example, one such cyclization reagent is represented by structure IIIa:

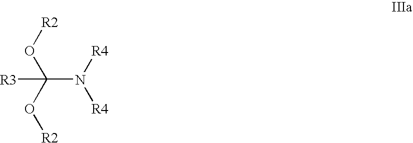

Each R2 is independently a substituted or unsubstituted aliphatic group, or a substituted or unsubstituted aryl group; or both R2 groups, taken together, are an inert linking group. When R3 is —H, R2 is preferably a secondary or tertiary alkyl group or a substituted or unsubstituted aryl group.

R3 is —H, a substituted or unsubstituted aliphatic group, a substituted or unsubstituted aryl group, or an electronegative or electropositive group. Preferably, R3 and R0 are both —H or a substituted or unsubstituted aliphatic group.

Each R4 is —H, a substituted or unsubstituted aliphatic group, a substituted or unsubstituted aryl group, or both R4 groups, taken together with the nitrogen atom to which they are bonded, are a substituted or unsubstituted heterocyclic group.

Another cyclization reagent is prepared by reacting a compound represented by structure IIIb with an alkylating agent.

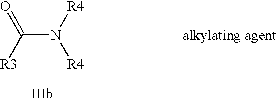

R3 and R4 are as defined above for IIIa.

The present invention is directed towards a method of preparing a product compound IIa by reacting a substrate IVa with one of the cyclization reagents defined above:

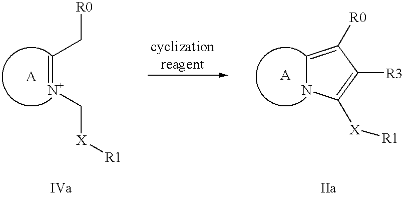

Ring A is a substituted or unsubstituted heteroaryl group.

X is a covalent bond, or a linking group selected from a methanone, a sulfone, a sulfoxide, a substituted or unsubstituted amine, or a substituted or unsubstituted methylene. Preferably, X is a linking group selected from a methanone, a sulfone, a sulfoxide, or a substituted or unsubstituted methylene. More preferably, X is a methanone.

R0 is —H, a substituted or unsubstituted aliphatic group, a substituted or unsubstituted aryl group, a substituted or unsubstituted non-aromatic heterocyclic group, a halogen, —CN, —COR^{a}, —CO₂R^{a}, —CONR^{a}R^{b}, —SO₂R^{a}, or —SO₂NR^{a}R^{b}.

R1 is —H, a substituted or unsubstituted aliphatic group, a substituted or unsubstituted aryl group, a substituted or unsubstituted non-aromatic heterocyclic group, —CN, —OR^{a}, —SR^{a}, or —NR^{a}R^{b}.

R3 is as described above for structure IIIa.

R^{a} and R^{b} are independently —H, alkyl, or aryl.

The advantages of the invention disclosed herein are significant. The improvements in the yield of the key cyclization step allow pharmacologically active indolizines, including the anticancer drugs disclosed in U.S. Provisional Application No. 60/322,020, to be made economically in pharmaceutically useful quantities. Furthermore, because this key step occurs early in the overall synthetic path, it enables the preparation of a wide range of structural variants that can be used in screening assays for other therapeutic targets. Finally, the higher yield and concomitant lack of byproduct formation leads to less waste, and thus an environmentally responsible process.

### DETAILED DESCRIPTION OF THE INVENTION

The methods disclosed herein can be used to prepare derivatives of nitrogen-containing polyaromatic systems, including indolizines, and in particular 3-acyl indolizines. The term indolizine refers to the two fused rings in structure I:

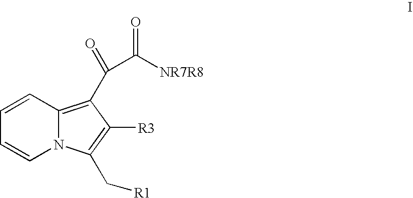

The method comprises the step of preparing a compound of structure IIa by a cyclization or ring forming reaction between the cyclization reagent and a substrate of structure IVa. One such cyclization reagent is IIIa. The other cyclization reagent is prepared by reacting IIIb with an alkylating agent. The variables in IIIa and IIIb are defined in the summary.

The cyclization reagent IIIa, in a molar ratio of 0.75 to 100 is combined with the substrate in a polar solvent and reacted at 70-170°. The polar solvent can be a polar protic solvent, such as water or an alcohol; a polar aprotic aromatic solvent such as nitrobenzene; or a polar aprotic solvent such as nitromethane, dimethyl acetamide (DMA), N,N-dimethyl formamide (DMF), dimethyl sulfoxide (DMSO), hexamethyl phosphoramide (HMPA), N-methylpyrrolidone (NMP), tetrahydrofuran (THF), or dioxane.

Alternatively, cyclization reagent IIIa, in a molar ratio of 0.75 to 100 is combined with the substrate in a polar solvent and reacted, the latter suspended or dissolved in a polar organic solvent such as an alcohol, nitrobenzene, nitromethane, DMA, DMF, DMSO, HMPA, NMP, THF, or dioxane. The resulting mixture is heated to between 100 to 160° C.

Preferably, cyclization reagent IIIa, in a molar excess of 5 to 15, is combined with the substrate in a solvent selected from DMA, DMF, DMSO, HMPA, NMP, nitrobenzene, nitromethane, or THF. The resulting mixture is heated to between 120 to 160° C.

Details of a specific preparation can be found in Example 2.

The cyclization reagent IIIb, in a molar excess of 2 to 100, and an alkylating agent, in a molar ratio of between 2 to 100, and the substrate, in a molar ratio of 1, are combined with a polar solvent and reacted at 25° to 70° C. The polar solvent can be a polar protic solvent, such as water or an alcohol; a polar aprotic aromatic solvent such as nitrobenzene; or a polar aprotic solvent such as nitromethane, DMA, DMF, DMSO, HMPA, NMP, THF, or dioxane, provided that said solvent is not a formamide different from IIIb. Subsequently, an excess of an amine is added and the mixture is stirred at 25 to 50° C.

Alternatively, the cyclization reagent IIIb, in a molar excess of between 2 to 20, is combined with an alkylating agent, in a molar excess of between 2 to 20, in a polar organic solvent, and stirred for 1 to 10 h at 30 to 70° C. The polar solvent can be an alcohol, nitrobenzene, nitromethane, DMA, DMF, DMSO, HMPA, NMP, THF, or dioxane, provided that said solvent is not a formamide different from IIIb. The result is combined with a solution of the substrate in said solvent, in a molar ratio of 1, and the mixture is reacted at 30 to 50° C. Subsequently, an excess of a trialkyl amine is added and the mixture is stirred at 30 to 50° C.

Preferably, the cyclization reagent IIIb, in a molar excess of 6 to 12, is combined with an alkylating agent, in a molar excess of between 6 to 12, in a polar organic solvent selected from the group of DMA, DMF, DMSO, HMPA, NMP, nitrobenzene, nitromethane, or THF, and reacted at 30 to 70° C., provided that said solvent is not a formamide different from IIIb. The result is combined with a solution of the substrate in said solvent, in a molar ratio of 1, and the mixture is reacted at 30 to 50° for between 45 to 75 minutes. Subsequently, an excess of triethyl amine is added and the mixture is stirred at 35 to 45° C.

Details of a specific preparation can be found in Example 1.

As noted previously, substituted indolizines prepared as detailed above can serve as starting materials for synthesizing 1-glyoxylamide indolizine such as I. Compounds represented by structure X can be prepared from compounds represented by structure IIc by acylation with, for example, oxalyl chloride or a synthetic equivalent thereof (e.g., oxalyl bromide):

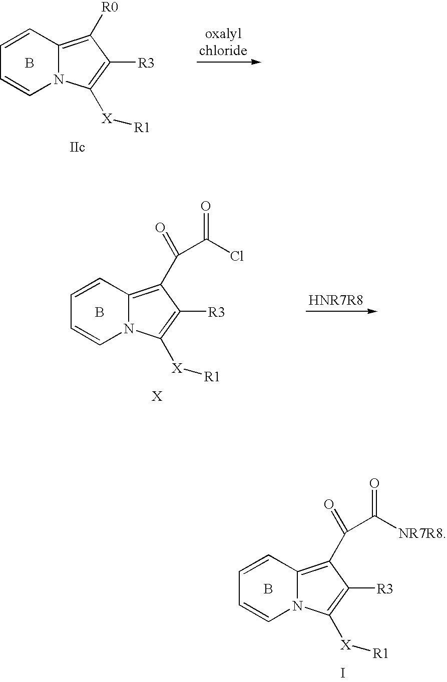

In the above scheme, R0 and R3 are —H and X, R7, R8 and Ring B are as described previously. Although equimolar amounts of an intermediates such as IIc and acylating agents can be used, typically the acylating agent is used in excess, for example, up to a twenty fold molar excess, preferably up to a ten fold molar excess and more preferably up to a three fold molar excess. Ethereal solvents (e.g., diethyl ether, tetrahydrofuran, 1,4-dioxane, glyme, diglyme and methyl tert-butyl ethyl) and aromatic solvents (e.g., benzene, toluene and xylene) are commonly used. Suitable reaction temperatures range from −50° C. to the boiling point of the solvent and more typically range from −10° C. to room temperature and preferably between −10° C. to 10° C. Detail of specific examples of this reaction are provided in U.S. Provisional Application No. 60/322,020, filed Sep. 13, 2001.

Compounds represented by structure X are converted into structure I by reacting the acylated intermediate with amine HNR7R8, wherein R7 and R8 are as described above. The acylated intermediate and the amine are mixed in a suitable solvent, e.g., an ethereal solvent or aromatic solvent. Suitable reaction temperatures are as described above for the acylation reaction. Although an excess of one reactant can be used (e.g., up to a ten-fold molar excess), more typically, between a 20% molar and 100% molar excess is used. When less than two equivalents of amine HNR₁R₂ are used, a tertiary amine such as triethylamine or dimethylaminopyridine is generally added so that at least two equivalents of amine compared to the acylated intermediate are present in the reaction mixture. Specific examples of this reaction are provided in U.S. Provisional Application No. 60/322,020, filed Sep. 13, 2001.

In a preferred embodiment, the variables in IIIa and IIIb are defined in the following paragraphs.

Each R2 is a substituted or unsubstituted cyclic aliphatic group, or —CH(R^{c})₂ or —C(R^{c})₃, and each R^{c} is independently a C1-C4 alkyl group. Preferably each R2 is independently —CH(CH₃)₂, —C(CH₃)₃, cyclobutyl, 2,2′,4,4′-tetramethylcyclobutyl, cyclopentyl, 2,2′,5,5′-tetramethlycyclopentyl, cyclohexyl, 2,2′,6,6′-tetramethlycyclohexyl, phenyl, or 2,6-dimethylphenyl.

R3 is as described above for structure IIIa. Preferably, R3 is —H, methyl, ethyl, or propyl. More preferably, R3 is —H.

Each R4 is —H, —CH₃, —CH₂CH₃, —CH₂CH₂CH₃, —CH(CH₃)₂ or —C(CH₃). Alternatively, both R4 groups, taken together with the nitrogen atom to which they are bonded, are a cyclic group as shown below:

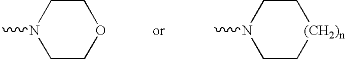

n is 0, 1, or 2.

In another preferred embodiment, the variables in IIIa and IIIb are defined in the following paragraphs.

Both R2 groups, taken together, are —(CR5₂)ₙ—, each R5 is independently —H or —CH₃.and n is 1, 2, or 3.

R3 and R4 are as described above for structure IIIa. Preferably, R3 is —H, methyl, ethyl, or propyl, and R4 is methyl, ethyl or propyl. More preferably, R3 is —H.

In yet another preferred embodiment, the cyclization reagent represented by IIIa is represented by V:

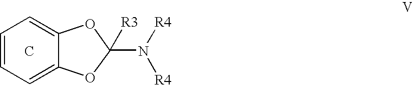

R3 and R4 are as described for structure IIIa. Preferably, R3 is —H, methyl, ethyl, or propyl, and R4 is methyl, ethyl or propyl. More preferably, R3 is —H.

Ring C is unsubstituted or substituted. More preferably, ring C is unsubstituted.

Most preferably, the cyclization reagent is N,N-dimethylformamide-di-tert-butyl acetal, N,N-dimethylacetamide-di-tert-butyl acetal, N,N-dimethylbenzamide-di-tert-butyl acetal, N,N-dimethylpropamide-di-tert-butyl acetal, or N,N-dimethyl-2-propamide-di-tert-butyl acetal; or is prepared by reacting N,N-dimethylformamide, N,N-dimethylacetamide, N,N-dimethylbenzamide, N,N-dimethylpropamide, or N,N-dimethyl-2-propamide with an alkylating agent.

The substrate used in the disclosed cyclization reaction is represented by structure IVa.

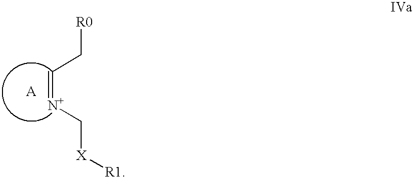

The reaction of a substrate of structure IVa with one of cyclization reagents disclosed herein results in the formation of a product of structure IIa. The variables in structures IIIa, IIIb and IVa are defined above. Preferably, R0 and R3 are both —H or a substituted or unsubstituted aliphatic group.

Preferably, the substrate is represented by structure VI:

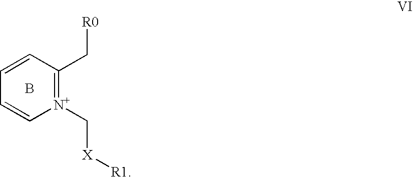

The reaction of a substrate of structure VI with one of the cyclization reagents disclosed herein results in the formation of a product represented by structure VII:

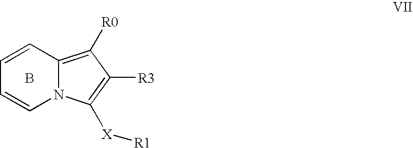

R0, R1, R3 and X in structures VI and VII are as described in structure IVa; and Ring B is substituted or unsubstituted. Suitable substituents for Ring B include those described below as being aryl ring substituents. Preferred substituents for Ring B include one or more groups selected from —F, —Cl, —Br, C1-C4 alkyl, C1-C4 alkoxy, —C1-C4 haloalkyl, C1-C4 haloalkoxy, —NH₂, —NO₂, or —CN. Preferably, however, Ring B is unsubstituted.

In a preferred embodiment, the substrate is represented by formula VIII:

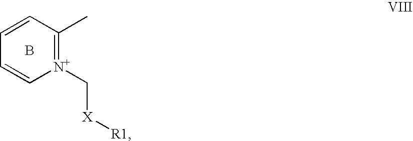

and R3 in the cyclization reagent is —H, resulting in the formation of a product represented by structure IX:

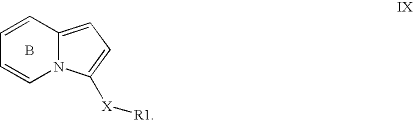

The variables in structure VIII and IX are as defined in structures VI and VII. Preferably, R1 is an optionally substituted phenyl, pyridyl, furanyl, thienyl, pyrazolyl, or pyrrolyl group (preferably phenyl group). Suitable substituents those described below as being aryl ring substituents. Preferably, the phenyl, pyridyl, furanyl, thienyl, pyrazolyl, or pyrrolyl group represented by R1 is substituted with zero, one or more substituents selected from —Br, —Cl, —F, —R^{a}, —OR^{a}, —CN, —COOR^{a}, —N(R^{a})₂, —CON(R^{a})₂, —NR^{a}COR^{b}, —NHCONH₂, or —SO₂N(R^{a})₂; and R^{a} and R^{b} are independently —H, an alkyl group or a substituted alkyl group. Especially preferred substitutents for a phenyl ring represented by R1 are —CH₃, —CH₂CH₃, —OCH₃, —CN, —F and —Cl, which are preferably at the para position relative to the methanone.

In structure I, variables X, R1 and R3 are as described for structure IVa; ring B is as defined for structure VI; and R7 and R8 are independently —H, a substituted or unsubstituted aliphatic group, a substituted or unsubstituted non-aromatic heterocyclic group, or a substituted or unsubstituted aryl group, provided that R7 or R8 are not both —H. Alternatively, NHR7R8, taken together, is a substituted or unsubstituted non-aromatic heterocyclic group, or a substituted or unsubstituted aryl group.

Preferably in structure I, X, R1 and R3 are as described for structure IVa; ring B is as defined for structure VI; R7 is —H; and R8 is a substituted or unsubstituted aliphatic group or a substituted or unsubstituted aryl group. Suitable values for R8 are in the section defining aryl groups. Commonly used aryl groups for R8 are selected from structural formulas i-xix below:

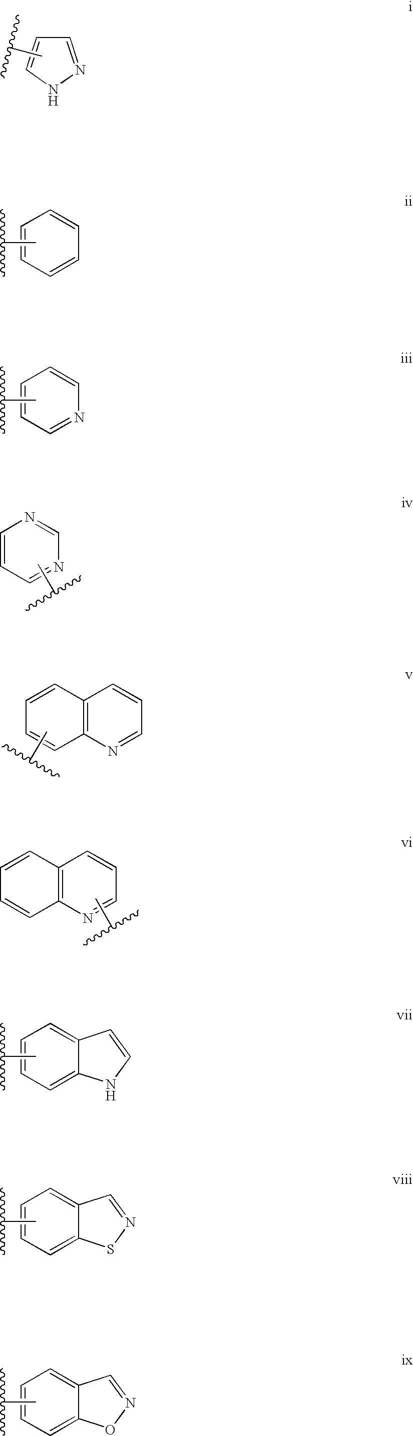

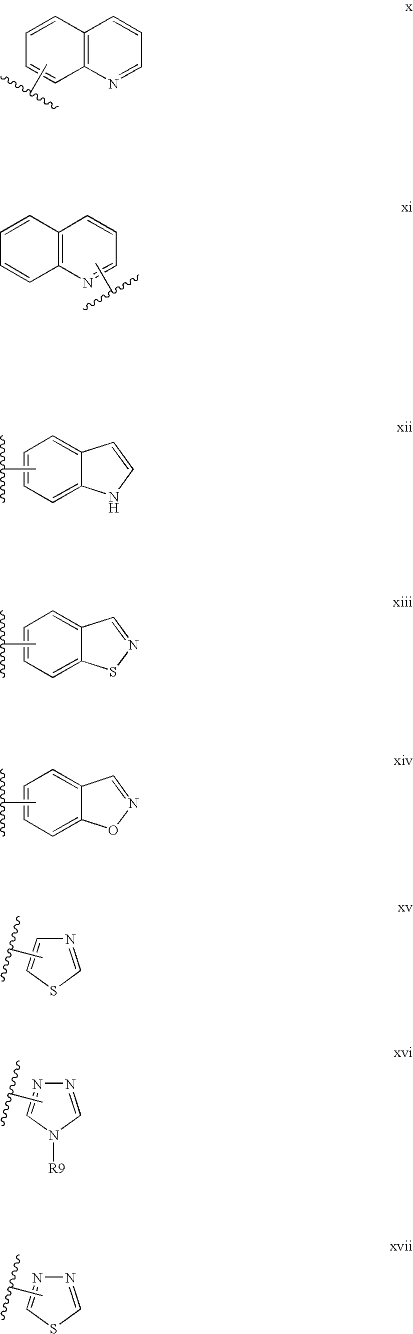

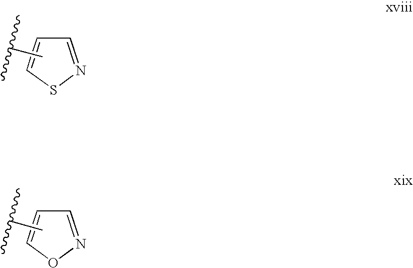

R9 is —H or a substituted or unsubstituted alkyl group. A more preferred value for R8 is a substituted or unsubstituted aryl group selected from structural formulas xx-xxv:

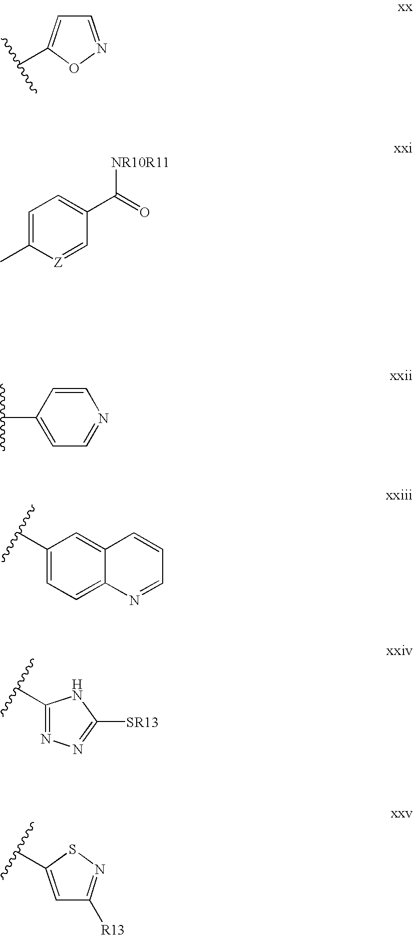

Z is —CH— or —N—; R10 and R 11 are independently —H or an alkyl group, or —NR10N11 taken together is a non-aromatic heterocyclic group; R12 is an alkyl group; and R13 is —H or an alkyl group. Structure xxv is a more preferred valued for R8 wherein R13 is —H, or a substituted or unsubstituted aliphatic group and preferably —CH₃.

An alkylating agent is a compound comprising an electrophilic alkyl group and a leaving group. Such agents are well-known to practitioners of the art. Examples include dialkyl sulfate or an alkyl mesylate, tosylate, triflate, chloride, bromide, or iodide. Preferably, the alkylating agent is dimethyl sulfate.

An inert linking group is any group that connects two other groups and does not substantially interfere with the reactions described herein. “Interfering with a reaction” refers to substantially decreasing the yield (e.g., a decrease of greater than 50%) or causing a substantial amount of by-product formation (e.g., where by-products represent at least 50% of the theoretical yield). Interfering substituents can be used, provided that they are first converted to a protected form. Suitable protecting groups are known in the art and are disclosed, for example, in Greene and Wuts, “Protective Groups in Organic Synthesis”, John Wiley & Sons (1991).

An aliphatic group is a straight chained, branched or cyclic (non-aromatic) hydrocarbon which is completely saturated or which contains one or more units of unsaturation. Typically, a straight chained or branched aliphatic group has from one to about twenty carbon atoms, preferably from one to about ten, and a cyclic aliphatic group has from three to about eight ring carbon atoms. An aliphatic group is preferably a completely saturated, straight-chained or branched alkyl group, e.g., methyl, ethyl, n-propyl, 2-propyl, n-butyl, sec-butyl, tert-butyl, pentyl, hexyl, pentyl or octyl, or a cycloalkyl group with three to about eight ring carbon atoms. C1-C20 straight chained and branched alkyl groups and C3-C8 cycloalkyl groups are also referred to herein as “lower alkyl groups”. Aliphatic groups may additionally be substituted or be interrupted by another group.

Aryl groups include carbocyclic aromatic groups such as phenyl, naphthyl, and anthracyl, and heteroaryl groups such as imidazolyl, isoimidazolyl, thienyl, furanyl, pyridyl, pyrimidyl, pyranyl, pyrrolyl, pyrazolyl, pyrazinyl, thiazolyl, isothiazolyl, oxazolyl, isooxazolyl, 1,2,3-triazolyl, 1,2,4-triazolyl, and tetrazolyl.

Aryl groups also include fused polycyclic aromatic ring systems in which a carbocyclic aromatic ring or heteroaryl ring is fused to one or more other heteroaryl rings. Examples include benzothienyl, benzofuranyl, indolyl, isoindolyl, quinolinyl, benzothiazolyl, benzoisothiazolyl, benzooxazolyl, benzoisooxazolyl, benzimidazolyl, indolizinyl, quinolinyl, and isoquinolinyl.

Non-aromatic heterocyclic rings are non-aromatic carbocyclic rings that include one or more heteroatoms such as nitrogen, oxygen or sulfur in the ring. The ring can be from three to about eight ring atoms. Examples include epoxyl, oxazolinyl, oxazolidinyl, thiazolinyl, thiazolidinyl, tetrahydrofuranyl, tetrahyrothienyl, morpholino, thiomorpholino, pyrrolidinyl, piperazinyl, and piperidinyl.

Suitable substituents on alkyl, aliphatic, aryl, or non-aromatic heterocyclic groups are those that do not substantially interfere with the reactions described herein. “Interfering with a reaction” refers to substantially decreasing the yield (e.g., a decrease of greater than 50%) or causing a substantial amount of by-product formation (e.g., where by-products represent at least 50% of the theoretical yield). Interfering substituents can be used, provided that they are first converted to a protected form. Suitable protecting groups are known in the art and are disclosed, for example, in Greene and Wuts, ibid. Suitable substituents on an alkyl, aliphatic, aryl, or non-aromatic heterocyclic groups include, for example, —OH, halogen (—Br, —Cl, —I and —F), —OR^{d}, —O—COR^{d}, —COR^{d}, —CN, —NO₂, —COOH, —SO₃H, —NH₂, —NHR^{d}, —N(R^{d}R^{e}), —COOR^{d}, —CHO, —CONH₂, —CONHR^{d}, —CON(R^{d}R^{e}), —NHCOR^{d}, —NRCOR^{d}, —NHCONH₂, NHCONR^{d}H, —NHCON(R^{d}R^{e}), —NR^{f}CONH₂, —NRCONR^{d}H, —NR^{f}CON(R^{d}R^{e}), —C(═NH)—NH₂, —C(═NH)—NHR^{d}, —C(═NH)—N(R^{d}R^{e}), —C(═NR^{f})—NH₂, —C(═NR^{f})—NHR^{d}, —C(═NR^{f})—N(R^{d}R^{e}), —NH—C(═NH)—NH₂, —NH—C(═NH)—NHR^{d}, —NH—C(═NH)—N(R^{d}R^{e}), —NH—C(═NR^{f})-NH₂, —NH—C(═NR^{f})—NHR^{d}, —NH—C(═NR^{f})—N(R^{d}R^{e}), NR^{g}H—C(═NH)—NH₂, —NR^{g}—C(═NH)—NHR^{d}, —NR^{g}—C(═NH)—N(R^{d}R^{e}), —NR^{g}—C(═NR^{f})—NH₂, —NR^{g}—C(═NR^{f})—NHR^{d}, —NR^{g}—C(═NR^{f})—N(R^{d}R^{e}), —NHNH₂, —SO₂NH₂, —SO₂NHR^{d}, —SO₂NR^{d}R^{e}, —CH═CHR^{d}, —CH═CR^{d}R^{e}, —CR^{f}═CR^{d}R^{e}, —CR^{f}═CHR^{d}, —CR^{f}═CR^{d}R^{e}, —CCR^{d}, —SH, —SOₖR^{d} (k is 0, 1 or 2) and —NH—C(═NH)—NH₂. R^{d}-R^{g} each are independently an aliphatic, substituted aliphatic, benzyl, substituted benzyl, aromatic or substituted aromatic group, preferably an alkyl, benzylic or aryl group. In addition, —NR^{d}R^{g}, taken together, can also form a substituted or unsubstituted non-aromatic heterocyclic group. A benzylic group, non-aromatic heterocyclic group or aryl group can also have an aliphatic or substituted aliphatic group as a substituent. A substituted alkyl or aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

Pharmacologically active indolizines disclosed elsewhere (WO 96/03383, WO 99/51605, WO 98/47507, WO99/24033, and WO 00/021,563) can also be prepared by combining the present invention with a suitable choice of starting materials.

Exemplification

The present invention is illustrated by the following examples, which are not intended to be limiting in any way.

### EXAMPLE 1

### New Cyclization Gives High Yield of Indolizine Intermediate and Reduced Byproducts: 4-(Indolizine-3-carbonyl)-benzonitrile

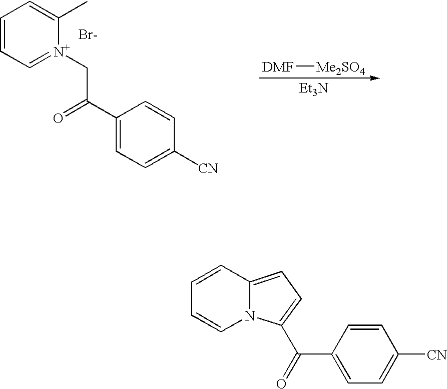

To 2-methyl-1-(4-cyano)-phenacyl pyridinium bromide (50 g, 120 mmol) DMF (500 mL) suspension solution was added DMF-Me₂SO₄ (400 mL, the mixture obtained by stirring a mixture of 1 eq. DMF and 1 eq Me₂SO₄ at 60° C. for 3 h, then allowing to rise to rt), and stirred at rt for 15 min. Subsequently, Et₃N (700 mL) was added and the mixture was stirred for 1 hr at ˜40° C. The mixture was then added to ice water (1200 mL), and the precipitate was collected, washed with water, and dried, to give 4-(indolizine-3-carbonyl)-benzonitrile (29 g, yield 76%). ¹H NMR (300 MHz, CDCl₃): 9.95 (d, 1H), 7.87-7.75(m, 4H), 7.57(d, 1H), 7.30-7.22(m, 2H), 6.97(m, 1H), 6.55(d, 1H); ESMS calcd for C₁₆H₁₀N₂ O: 246.08; Found: 247.1 (M+H)⁺.

### EXAMPLE 2

### New Cyclization Gives High Yield of Indolizine Intermediate and No Significant Byproducts: 4-(Indolizine-3-carbonyl)-benzonitrile

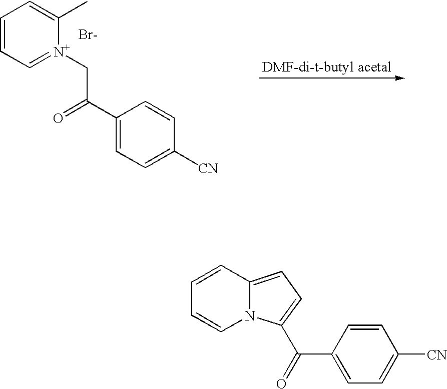

To 2-methyl-1-(4-cyano)-phenacylpyridinium bromide (5 g, 12.2 mmol) DMF (50 mL) suspension solution was added N,N-dimethylformamide di-t-butyl acetal (30 mL) at rt. The resulting clear solution was stirred at 130° C. for 4 min., then cooled to rt with an ice-water bath. Subsequently, water (100 mL) was added and the precipitate was collected and washed with water. Drying on a vacuum line gave 4-(indolizine-3-carbonyl)-benzonitrile (3.9 g, 90%) with 91% purity, which was crystallized with CH₃CN(35 mL) (82° C. to 0° C.) to give pure 2 (3.2 g). ¹H NMR (300 MHz, CDCl₃): 9.95 (d, 1H), 7.87-7.75(m, 4H), 7.57(d, 1H), 7.30-7.22(m, 2H), 6.97(m, 1H), 6.55(d, 1H); ESMS calcd for C₁₆H₁₀N₂ O: 246.08; Found: 247.1 (M+H)⁺.

### EXAMPLE 3

### Preparation of a Substrate: 4-indolizin-3-yl)-benzonitrile

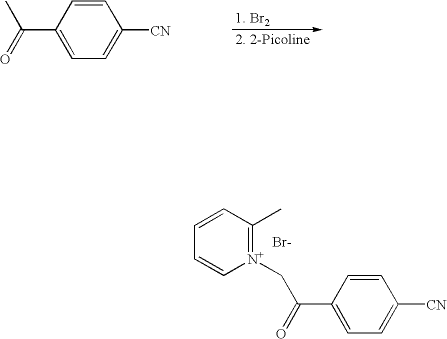

To 4-acetylbenzonitrile (14.5 g, 100 mmol) EtOAc (150 ml) solution was added Br₂ (5.1 ml, 100 mmol) at room temperature. The resulting mixture was stirred for 0.5 hr, and the solvent was evaporated under reduced pressure. The residue was dissolved in CH₃CN (100 ml), and picoline (20 ml, 200 mmol) was added to the mixture, which was then stirred for 30 minutes at room temperature and another 1 hr at 0° C. EtOAc (20 ml) was added to the mixture and the resulting precipitate was collected by filtration and washed with EtOAc to give pure 2-methyl-1-(4-cyno)-phenacylpyridinium bromide (20.3 g, 83%). ¹H NMR (300 MHz, DMSO): 9.05-8.03(m, 8H), 6.78(s, 2H), 2.74(s, 3H).

### Preparation of Other Compounds

The following compounds were prepared in 75% yield or greater, except as noted, using the methods of Examples 1 and 2. Analytical data and structural formulas are provided.

### EXAMPLE 4

### Indolizine-3-yl-phenyl-methanone

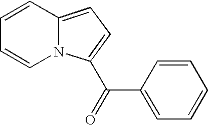

¹H-NMR (CDCl₃)δ(ppm), 9.98(d, J=6.9 Hz,1H),7.80(d, J=7.2 Hz, 2H), 7.59-7.45(m, 4H),7.35(d, J=4.8 Hz, 1H), 7.21(t, J=6.9 Hz, 1H), 6.95(t, J=6.6 Hz, 1H), 6.53(d, J=4.8 Hz, 1H); ESMS calcd for C₁₅H₁₁NO: 221.08; Found: 222.1 (M+H)⁺.

### EXAMPLE 5

### (4-Chloro-phenyl)-indolizin-3-yl-methanone

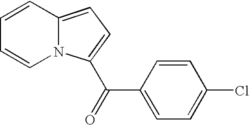

¹H-NMR (CDCl₃)δ(ppm), 9.94(d, J=7.2 Hz, 1H), 7.75(d, J=8.4 Hz, 2H), 7.57(d, J=9.0 Hz,1H), 7.46(d, J=8.4 Hz, 2H),7.30(d, J=4.5 Hz, 1H), 7.21(t, J=7.2 Hz, 1H), 6.95(t, J=6.9 Hz,1H), 6.53(d, J=4.5 Hz, 1H); ESMS calcd for C₁₅H₁₀ClNO: 255.05; Found: 256.0 (M+H)⁺.

### EXAMPLE 6

### (3,4-Dichloro-phenyl)-indolizin-3-yl-methanone

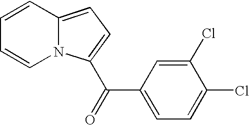

¹H-NMR (CDCl₃)δ(ppm), 9.94(d, J=7.2 Hz, 1H), 7.89(d, J=2.1 Hz, 1H), 7.65-7.55(m, 3H), 7.31(d, J=4.5 Hz, 1H), 7.27-7.21(m, 1H), 6.98(t, J=7.2 Hz, 1H), 6.56(d, J=4.8 Hz, 1H); ESMS calcd for C₁₅H₉Cl₂NO: 290.14; Found: 291.1(M+H)⁺.

### EXAMPLE 7

### Indolizin-3-yl-p-tolyl-methanone

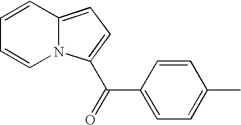

¹H-NMR (CDCl₃)δ(ppm), 9.92 (d, J=7.2, 11H), 7.71 (d, J=7.8, 2H), 7.43 (d, J=8.2, 1H), 7.32 (d, J=4.8, 1H), 7.24 (d, J=7.8, 2H), 7.08 (t, J=6.9, 1H), 6.81 (t, J=6.9, 1H), 6.42 (d, J=4.8, 1H). ESMS calcd for C₁₆H₁₁NO: 235.10; Found: 236.1 (M+H)⁺.

### EXAMPLE 8

### 4-Hydroxyphenyl-indolizin-3-yl-methanone

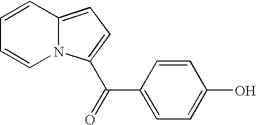

¹H-NMR (CDCl₃)δ(ppm), 9.83 (d, J=7.2, 1H), 7.74 (d, J=7.8, 2H), 7.59 (d, J=8.2, 1H), 7.40 (d, J=4.7, 1H), 7.19 (t, J=6.9, 2H), 6.97-6.87 (m, 3H), 6.81 (t, J=6.9, 1H), 6.55 (d, J=4.7, 1H). ESMS calcd for C₁₅H₁₁NO₂: 237.08; Found: 238.1 (M+H)⁺.

### EXAMPLE 9

### Indolizin-3-yl-(3-methoxy-phenyl)-methanone

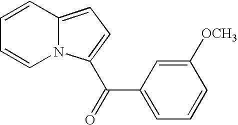

¹H-NMR (CDCl₃)δ(ppm), 9.96(d, J=7.2 Hz, 1H),7.54 (d, J=7.5 Hz, 1H), 7.39-7.33(m, 4H), 7.16(t, J=6.6 Hz, 1H), 7.08-7.04(m, 1H), 6.91(t, J=6.9 Hz, 1H), 6.50(d, J=4.5 Hz, 1H),3.85(s, 3H); ESMS calcd for C₁₆H₁₃NO₂:251.09; Found: 252.1 (M+H)⁺.

### EXAMPLE 10

### Indolizin-3-yl-(4-methoxy-phenyl)-methanone

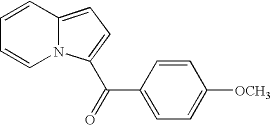

¹H-NMR (CDCl₃)δ(ppm), 9.9(d, J=6.9 Hz, 1H), 7.84-7.80(m, 2H), 7.53(d, J=9.0 Hz, 1H), 7.35(d, J=6.0 Hz, 1H), 7.13(t, J=8.1 Hz, 1H), 7.0-6.96(m, 2H),6.88(t, J=6.9 Hz, 1H), 6.51(d, J=4.5 Hz, 1H), 3.87(s, 3H); ESMS calcd for C₁₆H₁₃NO₂:251.09; Found: 252.1 (M+H)⁺.

### EXAMPLE 11

### 3-(Indolizine-3-carbonyl)-benzonitrile

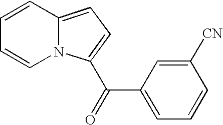

¹H-NMR (CDCl₃) δ (ppm), 9.95(d, J=7.2 Hz, 1H), 8.08-8.01(m, 2H), 7.81(d, J=7.8 Hz, 1H), 7.64-7.59(m, 2H), 7.29-7.24(m, 2H), 7.00(t, J=6.9 Hz, 1H), 6.57(d, J=4.8 Hz, 1H); ESMS calcd for C₁₆H₁₀N₂ O: 246.08; Found: 247.1 (M+H)⁺.

### EXAMPLE 12

### 4-(1-Methyl-indolizine-3-carbonyl)-benzonitrile

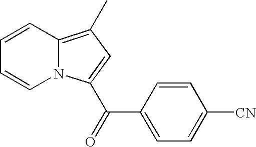

¹H-NMR (CDCl₃) δ (ppm), 9.96(d, J=7.2 Hz, 1H), 7.87-7.84(m, 2H), 7.79-7.76(m, 2H), 7.55(d, J=8.7 Hz, 1H), 7.27(t, J=6.0 Hz, 1H), 7.05(s, 1H), 6.99(t, J=6.9 Hz, 1H), 2.34(s, 3H); ESMS calcd for C₁₇H₁₂N₂O: 260.09; Found: 261.1 (M+H)⁺.

### EXAMPLE 13

### 4-(6-Ethyl-indolizine-3-carbonyl)-benzonotrile

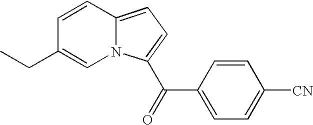

¹H-NMR (CDCl₃) δ (ppm), 9.84(d, J=0.9 Hz, 1H), 7.88-7.85(m, 2H), 7.79-7.76(m, 2H), 7.53(d, J=9.0 Hz, 1H), 7.19-7.16(m, 2H), 6.5(d, J=5.1 Hz, 1H),2.74(q, J=7.8 Hz, J=15.3 Hz, 2H), 1.33(t, J=7.2 Hz, 3H); ESMS calcd for C₁₈H₁₄N₂O: 274.11; Found: 275.1 (M+H)⁺.

### EXAMPLE 14

### 4-(6-Hydroxy-indolizine-3-carbonyl)-benzonitrile

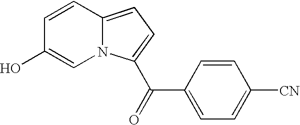

¹H-NMR (DMSO-d₆) δ (ppm), 9.94(s, 1H), 9.64(s, 1H), 8.00-7.98(m, 2H), 7.88-7.84(m, 2H), 7.73-7.69(m, 1H), 7.15-7.11(m, 2H), 6.61(d, J=4.8 Hz, 1H); ESMS calcd for C₁₆H₁₀N₂O₂:262.07; Found: 263.1 (M+H)⁺.

### EXAMPLE 15

### 4-(6-Methoxymethoxy-indolizine-3-carbonyl)-benzonitrile

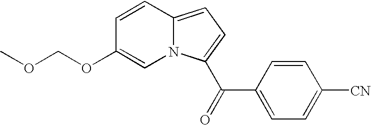

¹H-NMR (CDCl₃) δ (ppm), 9.92((s, 1H), 7.87(d, J=8.1 Hz,2H), 7.77(d, J=8.1 Hz, 2H), ESMS calcd for C₁₈H₁₄N₂O₃: 306.10; Found: 307.1 (M+H)⁺.

### EXAMPLE 16

### Indolizin-3-yl-(4-nitro-phenyl)-methanone

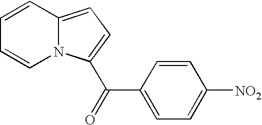

¹H-NMR (CDCl₃) δ (ppm), 9.97(d, J=6.6 Hz, 1H), 8.33(d, J=6.9 Hz, 2H), 7.92(d, J=6.9 Hz, 2H), 7.61(d, J=8.7 Hz, 1H), 7.30-7.25(m, 2H), 7.01(t, J=6.6 Hz, 1H), 6.57(d, J=3.0 Hz, 1H); ESMS calcd for C₁₅H₁₀N₂O₃: 266.07; Found: 267.0 (M+H)⁺.

### EXAMPLE 17

### (5-Chloro-thiophen-2-yl)-indolizin-3-yl-methanone

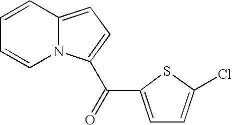

¹H-NMR (CDCl₃) δ (ppm), 9.79(d,J=7.2 Hz, 1H), 7.61(d,J=4.5 Hz, 1H), 7.55-7.50(m,2H), 7.15(t, J=7.5 Hz, 1H), 6.95(d,J=3.9 Hz, 1H), 6.88(t, J=7.2 Hz, 1H), 6.53(d, J=4.8 Hz, 1H); ESMS calcd for C₁₃H₈ClNOS: 261.00; Found: 262.0 (M+H)⁺.

### EXAMPLE 18

### 5-(Indolizine-3-carbonyl)-thiophene-2-carbonitrile

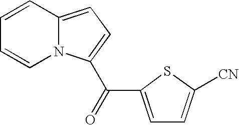

¹H-NMR (CDCl₃) δ (ppm), 9.88(d, J=6.9 Hz, 1H), 7.68-7.63(m,4H), 7.30-7.25(m, 1H), 7.00(t, J=6.9 Hz, 1H), 6.61(d, J=4.5 Hz, 1H); ESMS calcd for C₁₄H₈N₂OS: 252.04; Found: 253.0 (M+H)⁺.

### EXAMPLE 19

### Furan-2-yl-indolizin-3-yl-methanone

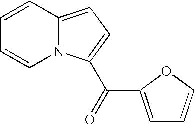

¹H-NMR (CDCl₃) δ (ppm), 10.01(d, J=7.2 Hz, 1H), 8.05(d, J=4.5 Hz, 1H), 7.63(s.1H), 6.60-6.56(m, 2H); ESMS calcd for C₁₃H₉NO₂: 211.06; Found: 212.1 (M+H)⁺.

### EXAMPLE 20

### 1-Indolizin-3yl-ethanone

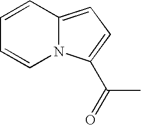

¹H-NMR (CDCl₃) δ (ppm), 9.84 (d, J=8.1, 1H), 7.47 (m, 2H), 7.07 (t, J=6.8, 1H), 6.82 (t, J=6.8, 1H), 6.47 (d, J=5.9, 1H), 2.54 (s, 3H). ESMS calcd for C₁₀H₉NO: 159.07; Found: 160.1 (M+H)⁺.

### EXAMPLE 21

### 1-Indolizin-3yl-propan-1-one

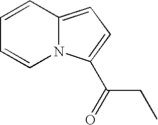

¹H-NMR (CDCl₃) δ (ppm), 9.89 (d, J=7.7, 1H), 7.49 (d, J=6.0, 2H), 7.08 (t, J=6.7, 1H), 6.82 (t, J=6.7, 1H), 6.47 (d, J=4.1, 1H), 2.91 (dd, J=10.1, 2H), 1.27 (t, J=10.1, 3H). ESMS calcd for C₁₁H₁₁NO: 173.08; Found: 174.1 (M+H)⁺.

### EXAMPLE 22

### 1-Indolizin-3yl-pentan-1-one

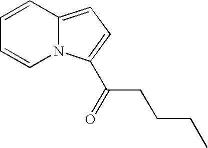

¹H-NMR (CDCl₃) δ (ppm), 9.88 (d, J=7.2, 1H), 7.51 (d, J=6.4, 2H), 7.13 (t, J=6.8, 1H), 6.82 (t, J=4.8, 1H), 6.48 (d, J=3.8, 1H), 2.83 (t, J=10.2, 2H), 1.76-1.42 (m, 4H), 0.94 (t, J=9.8, 3H). ESMS calcd for C₁₃H₁₅NO: 201.12; Found: 202.1 (M+H)⁺.

### EXAMPLE 23

### Indolizine-3-yl-phenyl-methanone

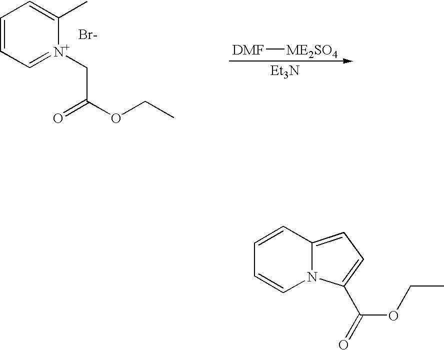

¹H NMR (300 MHz, CDCl₃), δ (ppm): 9.43 (dd, J=7.2 Hz, 0.6 Hz, 1H); 7.47-7.53 (m, 2H); 7.00 (m, 1H); 6.79 (m, 1H); 6.48 (d, J=3.9 Hz, 1H); 4.38 (q, J=7.2 Hz, 2H); 1.40 (t, J=7.2 Hz, 3H); 11% yield; ESMS calcd. for C₁₁H₁₂NO₂ (M+H)⁺: 190.1; Found: 190.1.

### EXAMPLE 24

### (7-Chloro-indolizin-3-yl)-(4-chloro-phenyl)-methanone

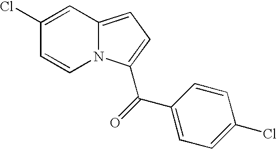

¹H NMR (300 MHz, CDCl3), δ (ppm): 9.85 (d, J=7.5 Hz, 1H); 7.73-7.75 (m, 2H); 7.55-7.56 (m, 1H); 7.45-7.48 (m, 2H); 7.32 (d, J=7.5 Hz, 1H); 6.91 (dd, J=7.5 Hz, 1.5 Hz, 1H); 6.49 (d, J=4.8 Hz, 1H); ESMS calcd. for C₁₅H₁₀Cl₂NO (M+H)⁻: 290.1; Found: 290.1.

### EXAMPLE 25

### (7-Chloro-indolizin-3-yl)-(4-cyano-phenyl)-methanone

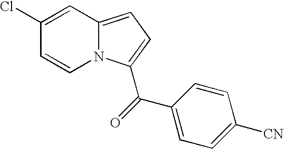

¹H NMR (300 MHz, CDCl3), δ (ppm): 9.88 (d, J=7.5 Hz, 1H); 7.78-7.88 (m, 4H); 7.59 (dd, J=7.5 Hz, 0.9 Hz, 1H); 7.26-7.28 (m, 1H); 6.96 (dd, J=7.5 Hz, 2.4 Hz, 1H); 6.52 (dd, J=7.5 Hz, 0.6 Hz, 1H); ESMS calcd. for C₁₆H₁₀ClN₂O (M+H)⁻: 281.0; Found: 281.0.

### EXAMPLE 26

### 3-(4-cyano-benzoyl)-indolizine-6-carboxylic acid methyl ester

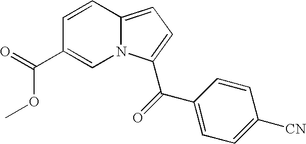

¹H-NMR (CDCl₃) δ (ppm) 10.60,s, 1H), 7.92-7.89(m, 2H), 7.82-7.77(m, 3H), 7.62(d, J=9.6 Hz, 1H), 7.38(d, J=6.3 Hz, 1H), 6.63(d, J=4.5 Hz, 1H), 3.99(s, 3H); ESMS clcd for C₁₈H₁₂N₂O₃: 304.08; Found: 305.1 (M+H)⁺.

### EXAMPLE 27

### 4-(indolizine-3-carbonyl)-benzoic acid ethyl ester

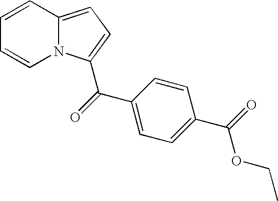

¹H-NMR (CDCl₃) δ (ppm), 9.98(d, J=6.6 Hz, 1H), 8.17-8.14(m, 2H), 7.85-7.82(m, 2H), 7.59(d, J=9.3 Hz, 1H), 7.30-7.20(m, 2H), 6.97(t, J=7.2 Hz, 1H), 6.54(d, J=4.8 Hz, 1H), 4.42(q, J=6.9 Hz, J=15 Hz, 2H), 1.43(t, J=7.2 Hz, 3H); ESMS clcd for C₁₈H₁₅NO₃: 293.11; Found: 294.2 (M+H)⁺.

### EXAMPLE 28

### Indolizin-3-yl-(4-nitro-phenyl)-methanone

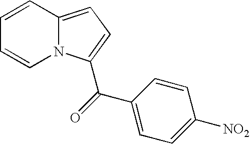

¹H-NMR (DMSO-d₆) δ 6.5 (m, 1H), 6.7 (m, 1H), 6.8 (d, 1H, J=5), 7.4 (d, 1H, J=5), 7.8 (d, 1H, J=5), 8.0 (d, 1H, J=5), 8.3 (d, 2H, J=8), 8.6 (d, 1H, J=8)ppm. ESMS calcd for C₁₅H₁₀N₂O₃: 266.1; Found: 267.1 (M+H)⁺.

### EXAMPLE 29

### 5-Methyl-indolizine-3-carboxylic acid tert-butyl ester

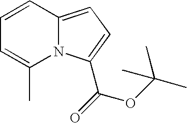

¹H-NMR (CDCl₃) δ 1.5 (s, 9H), 2.6 (s, 3H), 6.4 (d, 1H, J=4), 6.5 (d, 1H, J=8), 6.9 (dd, 1H, J, J=8, 8), 7.3 (d, 1H, J=8), 7.4 (d, 1H, J=5) ppm. ESMS calcd for C₁₄H₁₇NO₂: 231.1; Found: 232.1 (M+H)⁺.

### EXAMPLE 30

### (7-Fluoro-indolizin-3-yl)-(4-fluorophenyl)-methanone

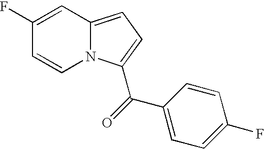

¹H NMR δ (DMSO-d₆) 9.96 (dd, J₁=5.4 Hz, J₂=7.8 Hz, 1H), 7.81 (dd, J₁=8.7 Hz, J₂=5.4 Hz, 2H), 7.34 (d, J=4.5 Hz, 1H), 7.14-7.20 (m, 2H), 6.49-6.81 (m, 3H), 6.48 (d, J=4.8 Hz, 1H); ESMS Calcd (C₁₅H₉F₂NO): 257.07, found 258.1 (M+H)⁺.

### EXAMPLE 31

### (4-Fluoro-phenyl)-(7-methoxy-indolizin-3-yl)-methanone

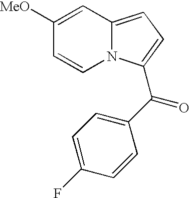

¹H-NMR (CDCl₃, 300 MHz): δ 9.83 (d, J=7.8 Hz, 1H), 7.82-7.77 (m, 2H), 7.25 (d, J=4.5 Hz, 1H), 7.17-7.12 (m, 2H), 6.82 (d, J=2.4 Hz, 1H), 6.65 (dd, J=2.4, 7.8 Hz, 1H), 6.34 (d, J=4.5 Hz, 1H), 3.89 (s, 3H, OCH₃); ES-MS: Calculated: C16H12FNO2 269.09, Found: 270.0 (M+H)⁺.

### EXAMPLE 32

### (7-Chloro-indolizin-3-yl)-(4-fluoro-phenyl)-methanone

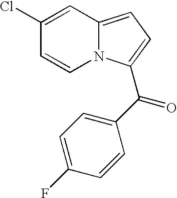

¹H-NMR (CDCl₃, 300 MHz): δ 9.85 (dt, J=0.6, 7.2 Hz, 1H), 7.84-7.79 (m, 2H), 7.56 (dd, J=0.6, 2.4 Hz, 1H), 7.33 (d, J=4.5 Hz, 1H), 7.20-7.14 (m, 2H), 6.90 (dd, J=2.1, 7.8 Hz, 1H), 6.49 (d, J=4.5 Hz, 1H); ES-MS: Calculated: C15H9ClFNO: 273.04, Found: 274.0 (M+H)⁺.

### EXAMPLE 33

### (4-Chloro-phenyl)-(7-methoxy-indolizin-3-yl)-methanone

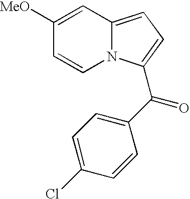

¹H-NMR (CDCl₃, 300 MHz): δ 9.85 (d, J=7.8 Hz, 1H), 7.74-7.71 (m, 2H), 7.46-7.42 (m, 2H), 7.24 (d, J=4.2 Hz, 1H), 6.83 (d, J=2.4 Hz, 1H), 6.66 (dd, J=2.7, 7.8 Hz, 1H), 6.35 (d, J=4.8 Hz, 1H), 3.89 (s, 3H); ES-MS: Calculated: C16H12ClNO2: 285.06, Found: 286.0 (M+H)⁺.

### EXAMPLE 34

### (4-Chloro-phenyl)-(7-methoxy-indolizin-3-yl)-methanone

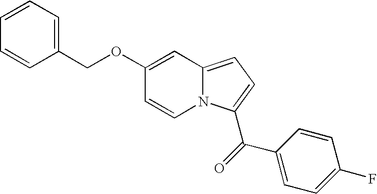

(7-Benyloxy-indolizin-3-yl)-(4-fluoro-phenyl)-methanone

¹H-NMR (CDCl₃) δ (ppm), 9.82 (d,J=12, 1H), 7.79-6.65(m, 12H), 6.32(d, J=5, 1H), 5.14 (s, 2H). ESMS clcd for C₂₂H₁₆FNO₂: 345.12; Found: 346.2 (M+H)⁺.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of preparing a compound represented by structural formula IIa: 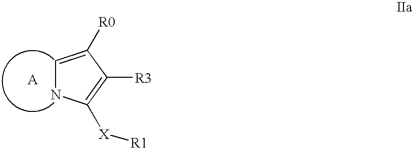
wherein ring A an is unsubstituted or substituted aryl group; comprising reacting a compound represented by structural formula IVa: 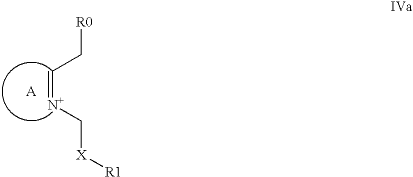
with either a compound represented by structural formula IIIa: 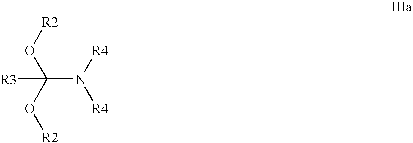
or, a reagent prepared by reacting the compound represented by structural formula IIIb with an alkylating agent: 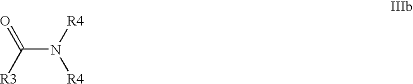
wherein:
X is a covalent bond, or a linking group selected from a methanone, a sulfone, a sulfoxide, a substituted or unsubstituted amine, or a substituted or unsubstituted methylene;
R0 is —H, a substituted or unsubstituted aliphatic group, a substituted or unsubstituted aryl group, a substituted or unsubstituted non-aromatic heterocyclic group, a halogen, —CN, —COR^{a}, —CO₂R^{a}, —CONR^{a}R^{b}, —SO₂R^{a}, or —SO₂NR^{a}R^{b};
R1 is —H, a substituted or unsubstituted aliphatic group, a substituted or unsubstituted aryl group, a substituted or unsubstituted non-aromatic heterocyclic group, —CN, —OR^{a}, —SR^{a}, or NR^{a}R^{b};
each R2 is independently a substituted or unsubstituted aliphatic group, or a substituted or unsubstituted aryl group; or both R2 groups, taken together, are an inert linking group;
R3 is —H, a substituted or unsubstituted aliphatic group, a substituted or unsubstituted aryl group, or an electron-withdrawing or electron-donating group, provided that if R3 is —H, at least one of R2 is a secondary or tertiary alkyl group, or a substituted or unsubstituted aryl group;
each R4 is independently —H, a substituted or unsubstituted aliphatic group, a substituted or unsubstituted aryl group;
or both R4 groups, taken together with the nitrogen atom to which they are bonded, are a substituted or unsubstituted heterocyclic group;
wherein R^{a} and R^{b} are independently —H, alkyl, or aryl.

2. The method of claim 1 wherein X is a covalent bond, or a linking group selected from a methanone, a sulfone, or a sulfoxide.

3. The method of claim 1 wherein R0 and R3 are independently —H, or a substituted or unsubstituted aliphatic group.

4. The method of claim 3 wherein if R3 is —H, at least one of R2 is a secondary or tertiary alkyl group, or a substituted or unsubstituted aryl group.

5. The method of claim 1 wherein X is methanone.

6. The method of claim 4 wherein:
a. R2 is a substituted or unsubstituted cyclic aliphatic group, or —CH(R^{c})₂, —C(R^{c})₃, and each R^{c} is independently a C1-C4 alkyl group; and
b. each R4 is —H, —CH₃, —CH₂CH₃, —CH₂CH₂CH₃, —CH(CH₃)₂—C(CH₃)₃, phenyl; or both R4 groups, taken together with the nitrogen atom to which they are bonded, are a cyclic group as shown below: 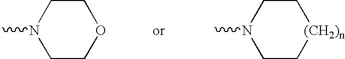
wherein n is 0, 1, or 2.

7. A method of preparing a compound represented by structural formula IIb: 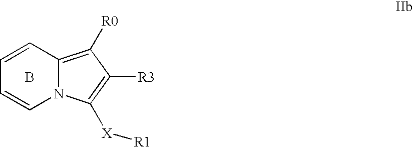
wherein ring B is unsubstituted or substituted or is fused to an aryl group; comprising reacting a compound represented by structural formula IVb: 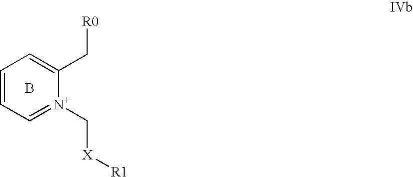
with either a compound represented by structural formula IIIa: 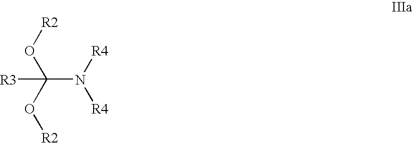
or, a reagent prepared by reacting the compound represented by structural formula IIIb with an alkylating agent: 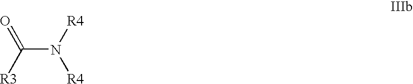
wherein:
X is a covalent bond, or a linking group selected from a methanone, a sulfone, a sulfoxide, a substituted or unsubstituted amine, or a substituted or unsubstituted methylene;
R0 is —H, a substituted or unsubstituted aliphatic group, a substituted or unsubstituted aryl group, a substituted or unsubstituted non-aromatic heterocyclic group, a halogen, —CN, —COR^{a}, —CO₂R^{a}, —CONR^{a}R^{b}, —SO₂R^{a}, or —SO₂NR^{a}R^{b};
R1 is —H, a substituted or unsubstituted aliphatic group, a substituted or unsubstituted aryl group, a substituted or unsubstituted non-aromatic heterocyclic group, —CN, —OR^{a}, —SR^{a}, or —NR^{a}R^{b};
each R2 is independently a substituted or unsubstituted aliphatic group, or a substituted or unsubstituted aryl group; or both R2 groups, taken together, are an inert linking group;
R3 is —H, a substituted or unsubstituted aliphatic group, a substituted or unsubstituted aryl group, or an electron-withdrawing or electron-donating group, provided that if R3 is —H, at least one of R2 is a secondary or tertiary alkyl group, or a substituted or unsubstituted aryl group;
each R4 is independently —H, a substituted or unsubstituted aliphatic group, or a substituted or unsubstituted aryl group;
or both R4 groups, taken together with the nitrogen atom to which they are bonded, are a substituted or unsubstituted heterocyclic group;
wherein R^{a} and R^{b} are independently —H, alkyl, or aryl.

8. The method of claim 7 wherein X is methanone, sulfone, or sulfoxide.

9. The method of claim 7 wherein:
a. R2 is a substituted or unsubstituted cyclic aliphatic group, or a substituted or unsubstituted pheyl group, or —CH(R^{c})₂ or —C(R^{c})₃, where each R^{c} is independently a C1-C4 alkyl group; and
b. each R4 is —H, —CH₃, —CH₂CH₃, —CH₂CH₂CH₃, —CH(CH₃)₂—C(CH₃)₃, phenyl; or both R4 groups, taken together with the nitrogen atom to which they are bonded, are a cyclic group as shown below: 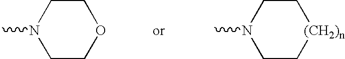
wherein n is 0, 1, or 2.

10. The method of claim 9 wherein each R2 is independently —CH(CH₃)₂, —C(CH₃)₃, cyclobutyl, 2,2′,4,4′-tetramethylcyclobutyl, cyclopentyl, 2,2′,5,5′-tetramethlycyclopentyl, cyclohexyl, 2,2′,6,6′-tetramethlycyclohexyl, phenyl, or 2,6-dimethylphenyl.

11. The method of claim 7 wherein both R2 groups, taken together, are —(CR5₂)ₙ— and n is 1, 2, or 3 and each R5 is independently —H or —CH₃.

12. The method of claim 7 wherein both R2, taken together, are represented by ring C: 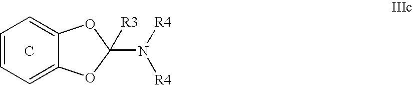
and wherein ring C is unsubstituted or substituted.

13. The method of claim 12 wherein ring C is unsubstituted.

14. The method of claim 7 wherein R2 is —C(CH₃)₃.

15. The method of claim 7 wherein R4 is —CH₃.

16. A method of preparing a compound represented by structural formula IIb: 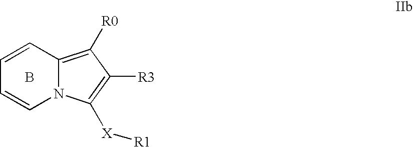
wherein ring B is unsubstituted or substituted or is fused to an aryl group; comprising reacting a compound represented by structural formula IVb: 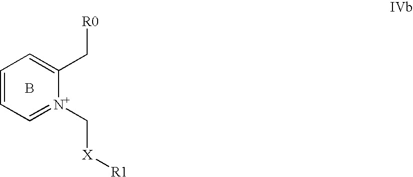
with either a compound represented by structural formula IIIa: 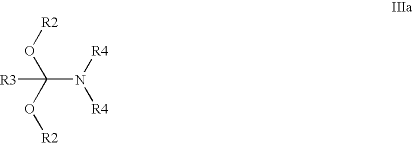
or, a reagent prepared by reacting the compound represented by structural formula IIIb with dimethyl sulfate: 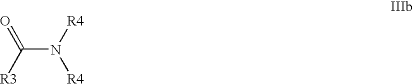
wherein:
X is a methanone, a sulfone, or a sulfoxide;
R0 is —H, a substituted or unsubstituted aliphatic group, a substituted or unsubstituted aryl group, a substituted or unsubstituted non-aromatic heterocyclic group, a halogen, —CN, —COR^{a}, —CO₂R^{a}, CONR^{a}R^{b}, —SO₂R^{a}, or —SO₂NR^{a}R^{b};
R1 is —H, a substituted or unsubstituted aliphatic group, a substituted or unsubstituted aryl group, a substituted or unsubstituted non-aromatic heterocyclic group, —CN, —OR^{a}, —SR^{a}, or —NR^{a}R^{b};
each R2 is independently —CH(R^{c})₂ or —C(R^{c})₃;
R3 is —H, or a substituted or unsubstituted aliphatic group; and
each R4 is —H, —CH₃, —CH₂CH₃, —CH₂CH₂CH₃, —CH(CH₃)₂—C(CH₃)₃, phenyl, or both R4 groups, taken together with the nitrogen atom to which they are bonded, are a cyclic group as shown below: 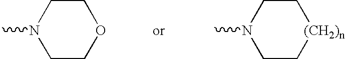
wherein n is 0, 1, or 2;
R^{a} and R^{b} are independently —H, alkyl, or aryl; and
each R^{c} is independently a C1-C4 alkyl group.

17. The method of claim 16 wherein each R2 is —C(CH₃)₃.

18. The method of claim 16 wherein each R4 is —CH₃.

19. The method of claim 18 wherein R0 and R3 are both —H.

20. The method of claim 18 wherein ring B is optionally substituted with one or more groups selected from —F, —Cl, —Br, C1-C4 alkyl, C1-C4 alkoxy, —C1-C4 haloalkyl, C1-C4 haloalkoxy, —NH₂, —NO₂, or —CN.

21. The method of claim 18 wherein ring B is unsubstituted and R1 is a phenyl, pyridyl, furanyl, thienyl, pyrazolyl, or pyrrolyl group substituted with zero, one or more substituents selected from: —Br, —Cl, —F, —R^{a}, —OR^{a}, —CN, —COOR^{a}, —N(R^{a})₂, —CON(R^{a})₂, —NR^{a}COR^{b}, —NHCONH₂, or —SO₂N(R^{a})₂.

22. The method of claim 19 wherein the compound represented by structural formula IIb is further reacted with oxalyl chloride or a synthetic equivalent thereof to form a first intermediate; and reacting the first intermediate with NHR7R8 to form a compound represented by structural formula I; 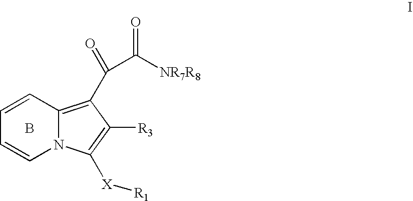
wherein R7 and R8 are independently —H, a substituted or unsubstituted aliphatic group, a substituted or unsubstituted non-aromatic heterocyclic group, or a substituted or unsubstituted aryl group, provided that R7 or R8 are not both —H, or NHR7R8, taken together, is a substituted or unsubstituted non-aromatic heterocyclic group, or a substituted or unsubstituted aryl group.

23. The method of claim 22 wherein R7 is H and R8 is represented by a structural formula selected from: 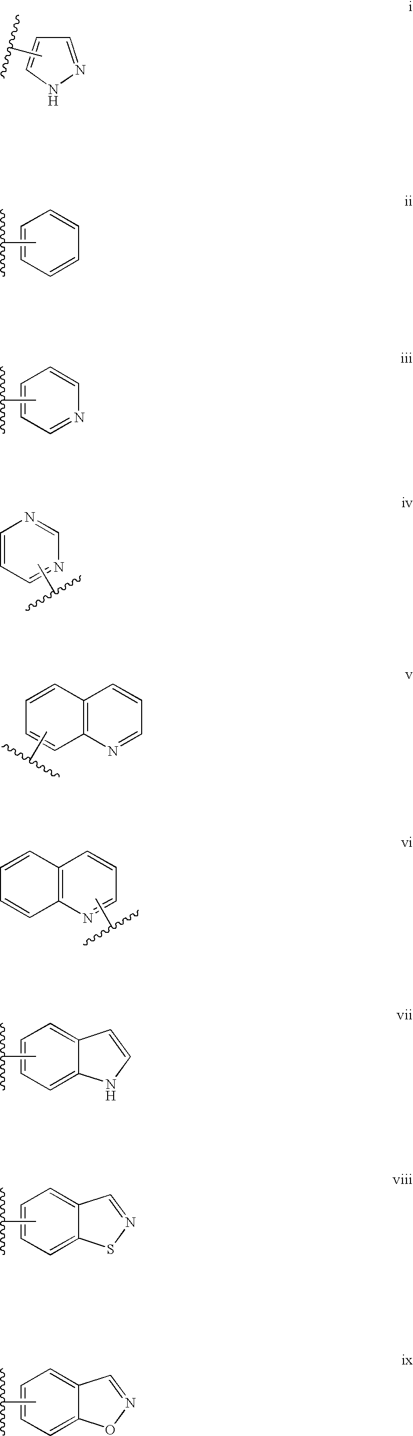 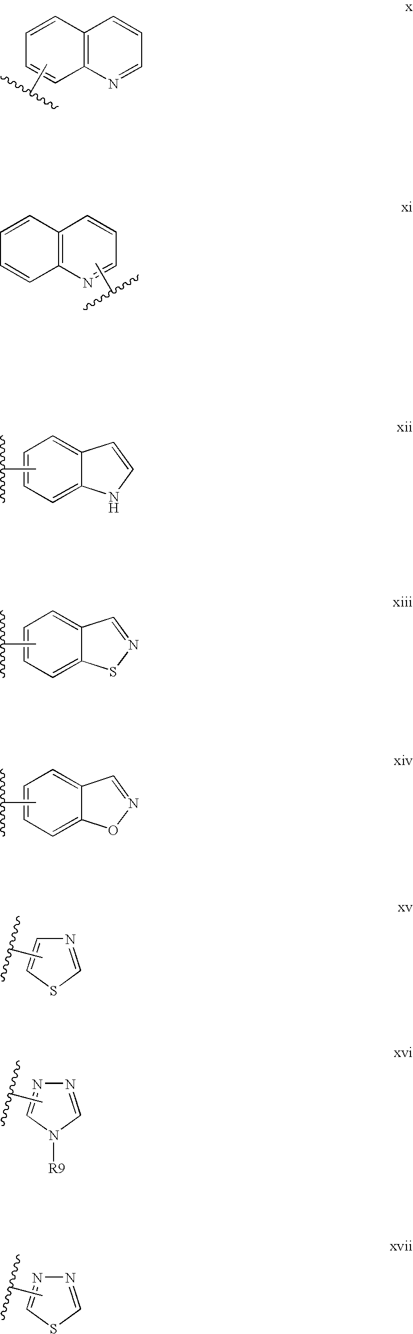 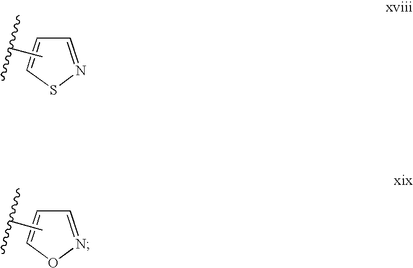
wherein R9 is —H or a substituted or unsubstituted alkyl group.

24. The method of claim 23 wherein R8 is represented by a structural formula selected from: 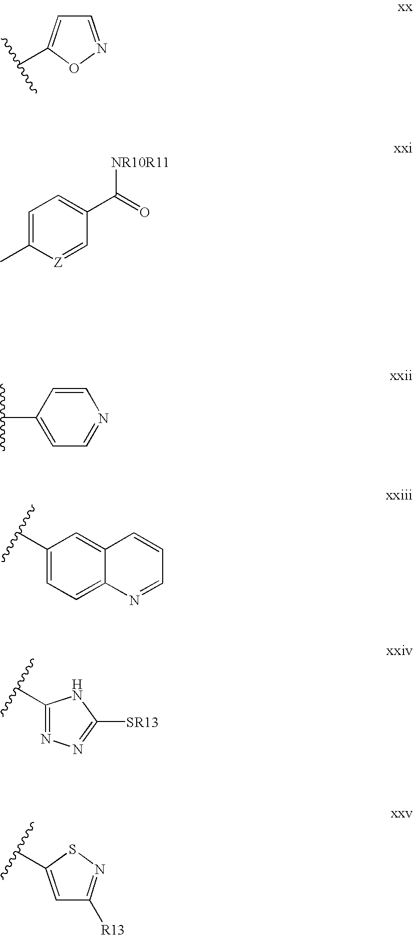
wherein Z is —CH— or —N—; R10 and R11 are independently —H or an alkyl group, or —NR10N11 taken together is a non-aromatic heterocyclic group; and R13 is —H or an alkyl group.

25. A method of preparing a compound represented by structural formula VII: 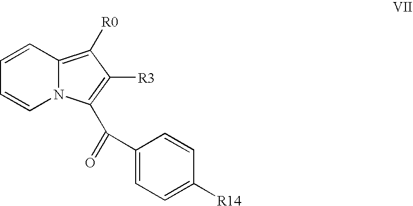
comprising reacting a compound represented by structural formula VIII: 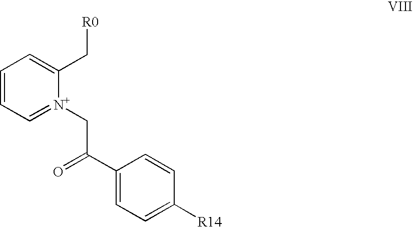
with either a compound represented by structural formula IIIa: 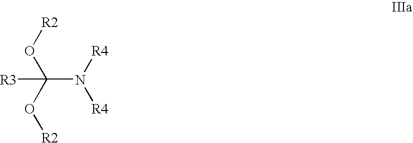
or, a reagent prepared by reacting the compound represented by structural formula IIIb with an alkylating agent: 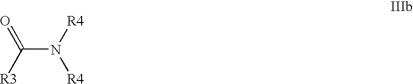
wherein
R2 is —C(CH₃)₃;
R0 and R3 are —H;
R4 is —CH₃; and
R14 is —CH₃, CH₂CH₃, —OCH₃, —CN, —F or —Cl.

26. The method of claim 25 wherein the compound represented by structural formula VII is further reacted with oxalyl chloride or a synthetic equivalent thereof to form a first intermediate; and reacting the first intermediate with NHR7R8 to form a compound represented by the following structural formula; 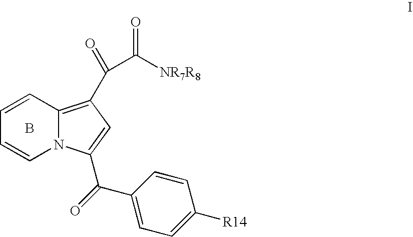
wherein R7 and R8 are independently —H, a substituted or unsubstituted aliphatic group, a substituted or unsubstituted non-aromatic heterocyclic group, or a substituted or unsubstituted aryl group, provided that R7 or R8 are not both —H, or NHR7R8, taken together, is a substituted or unsubstituted non-aromatic heterocyclic group, or a substituted or unsubstituted aryl group.

27. The method of claim 26 wherein R8 is represented by a structural formula selected from: 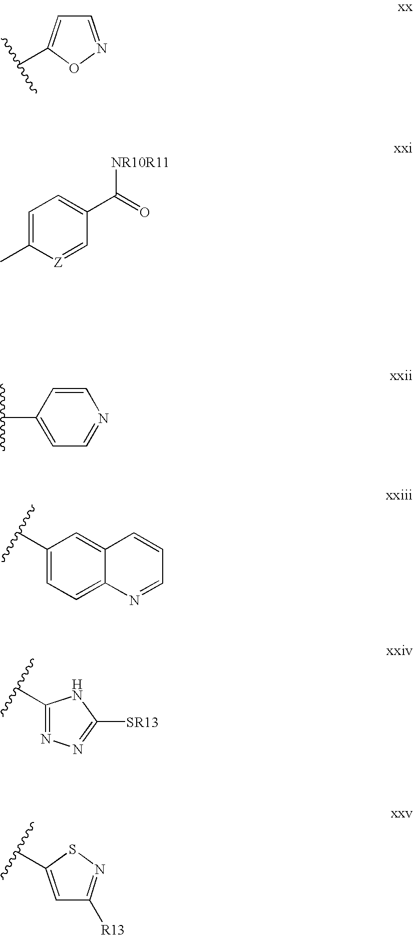
wherein Z is —CH— or —N—; R10 and R 11 are independently —H or an alkyl group, or —NR10N11 taken together is a non-aromatic heterocyclic group; R12 is an alkyl group; and R13 is —H or an alkyl group.

28. The method of claim 27 wherein R8 is represented by structural formula xxv and R13 is methyl.

29. The method of claim 28 wherein R14 is —CN.

30. The method of claim 7 wherein R0 and R3 are H, further comprising the steps of reacting the compound represented by structural formula IIb with oxalyl chloride or a synthetic equivalent thereof to form a first intermediate; and reacting the first intermediate with NHR7R8 to form a compound represented by structural formula I; 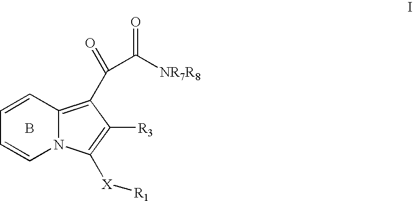
wherein R7 and R8 are independently —H, a substituted or unsubstituted aliphatic group, a substituted or unsubstituted non-aromatic heterocyclic group, or a substituted or unsubstituted aryl group, provided that R7 or R8 are not both —H, or NHR7R8, taken together, is a substituted or unsubstituted non-aromatic heterocyclic group, or a substituted or unsubstituted aryl group.
